(19) United States
(12) Patent Application Publication
(10) Pub. No.: US 2024/0374126 A1
(43) Pub. Date: Nov. 14, 2024

(54) CLEANING OF A MEDICAL DEVICE

(71) Applicant: Ecolab USA Inc., Saint Paul, MN (US)

(72) Inventors: Jean-Noël BERTHO, Sainghin en Mélantois (FR); Pierre BESSON, Sainghin en Mélantois (FR); Stéphane CLERQUIN, Sainghin en Mélantois (FR); Gaspard LAMERAND, Sainghin en Mélantois (FR); Sébastien TERRIER, Sainghin en Mélantois (FR)

(21) Appl. No.: 18/659,985

(22) Filed: May 9, 2024

Related U.S. Application Data
(60) Provisional application No. 63/501,274, filed on May 10, 2023.

(51) Int. Cl. A61B 1/12

(52) U.S. Cl. CPC A61B 1/123; A61B 1/125

(57) ABSTRACT

A method is described for cleaning a medical device. The method comprises coupling a lumen of the medical device to a cleaning machine and placing the medical device in a tank. The method also comprises injecting a first volume of a first fluid from the cleaning machine, through the coupled lumen and into the tank for preliminary flushing of the lumen and, following the injection of the first volume of the first fluid, injecting a second fluid. Following the injection of the second fluid, the method comprises letting the second fluid sit in the lumen during a contact time period, and injecting a second volume of the first fluid for rinsing the second fluid from the lumen. Following the contact time period, the method also comprises injecting a gas through the lumen and, following the rinsing, maintaining the medical device in the tank during a soaking time.

### RELATED APPLICATION

This application claims the benefit of U.S. Provisional Application No. 63/501,274, filed on May 10, 2023, the entire contents of which are incorporated herein by reference.

### BACKGROUND

Medical devices such as endoscopes may be cleaned, for example in order to avoid soiling or to permit reusing such medical devices. In order to ease cleaning, cleaning processes have been developed to reduce or avoid the intervention of a human operator.

### SUMMARY

Cleaning medical device, such as endoscopes for example, may comprise manually brushing by healthcare professionals. Such a manual task may lead to producing debris, bioburden or biofilms outside of a cleaning device channel. Such manual tasks may be time consuming, involve infection risks, musculoskeletal risks and waste management. Example methods hereby described aim at reducing such drawbacks. In some example methods hereby described, brushing of lumens of internal channels of medical devices may be reduced or avoided. Such brushing that may be reduced or eliminated can involve passing a brush having bristles partially or fully through the length of the lumen being brushed and/or brushing one or more external surfaces of the medical device. Some example methods described herein involve preliminarily cleaning a medical device to remove some but not all soil and/or waste on and/or in the medical device followed by processing of the medical device in an automated reprocessing machine without brushing the medical device or brushing the medical device less than would otherwise be performed.

In one example, a method for cleaning a medical device is described. The method includes coupling a lumen of the medical device to a cleaning machine, thereby providing a coupled lumen, and placing the medical device in a tank. The method also includes injecting a first volume of a first fluid from the cleaning machine, through the coupled lumen and into the tank for preliminary flushing of the lumen and, following the injection of the first volume of the first fluid, injecting a second fluid from the cleaning machine, through the coupled lumen and into the tank, whereby the second fluid differs from the first fluid. The method additionally involves, following the injection of the second fluid, letting the second fluid sit in the lumen during a contact time period and, following the contact time period in which the second fluid sits in the lumen, injecting a second volume of the first fluid from the cleaning machine, through the coupled lumen and into the tank for rinsing the second fluid from the lumen. The method also includes, following the contact time period in which the second fluid sits in the lumen, injecting a gas through the lumen, and following the rinsing, maintaining the medical device in the tank during a soaking time, the tank containing a mixture of the first fluid and the second fluid.

In another example, a composition for cleaning of a medical device is described. The composition is in the form an aqueous solution comprising hydrogen peroxide, where the composition has a pH of less than 8. For example, the composition may include at least 5% of hydrogen peroxide, between 1 and 10% of surfactant, between 1 and 10% of sequestrant, between 1 and 10% of adjusting pH agent, and between 0 and 20% of fluidifying agent.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a chart of an example method.

FIG. 2 is chart of another example method.

FIG. 3 is a chart of another example method.

FIG. 4 is a block diagram of an example cleaning machine.

FIG. 5 is a block diagram of another example cleaning machine.

FIG. 6 is a block diagram of another example cleaning machine.

FIG. 7 is a block diagram of another example cleaning machine.

FIG. 8 is a block diagram of another example cleaning machine.

FIG. 9 is a block diagram of another example cleaning machine.

FIG. 10 represents an example viscosity of an example composition that can be used as an example second fluid.

### DETAILED DESCRIPTION

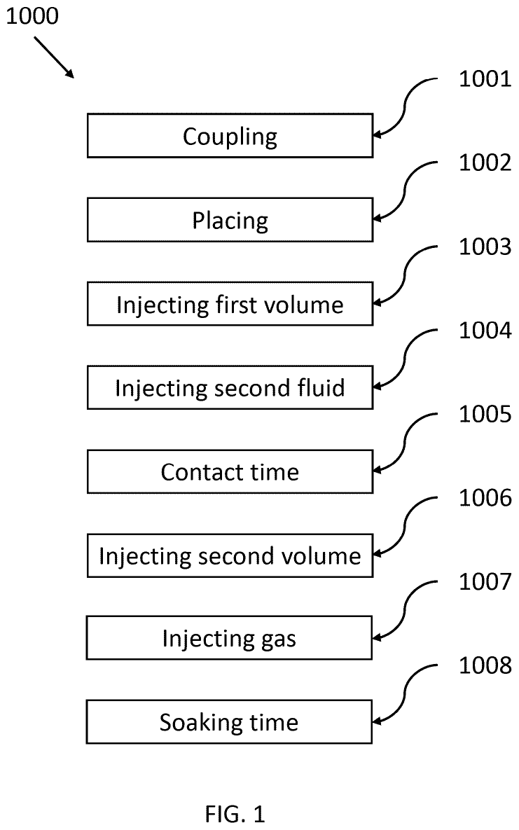

FIG. 1 represents an example method 1000 for cleaning a medical device. Cleaning according to the disclosure can be understood as removing soil and/or waste. In some examples, cleaning comprises removing solid soil or waste. In some examples, cleaning comprises removing fluid (e.g., liquid) soil and/or waste. In some examples, cleaning comprises removing a mixture of solid and fluid soil and/or waste. In some examples, the soil or waste comprises physiological soil or waste. The cleaning may be partial whereby the removal of soil and/or waste may be a partial removal, whereby some amount of soil and/or waste remain present on or in the medical device after cleaning. The solid soil and/or waste may coat an internal and/or external surface of the medical device prior to cleaning. The cleaning should be understood as removing at least some soil and/or waste.

The medical device being cleaned may be a mechanical or electromechanical apparatus used in medical procedure and brought in contact with physiological organs, parts, solids or fluids during use. Some example medical devices are endoscopes. Medical devices can have one or more conducts or channels, whereby such conduct or channels comprise an elongated internal cavity or lumen. Such a lumen may be exposed to physiological fluid circulation or to the circulation of a mixture of physiological fluid and solids during use. In some examples, such medical device comprises an identification tag which may be electromagnetically interrogated, for example in order to permit identifying and tracking such a medical device. Such identification tag may be an RFID (Radio Frequency Identification) tag.

As illustrated in block 1001, example method 1000 comprises coupling a lumen of the medical device to a cleaning machine. The coupling may be a mechanical fluidic coupling whereby a fluid may pass from the cleaning machine into the lumen as a result of the coupling. The coupling may be automated, may be as a result of human intervention, or may be as a result of both automation and human intervention. The coupling may be a direct coupling or an indirect coupling. A direct coupling should be understood as a coupling whereby a part of the medical device defining the lumen may be directly connected to a matching part of the cleaning machine without an intervening component. An indirect coupling should be understood as a coupling whereby one or more additional mechanical elements are provided between the cleaning machine and the lumen of the medical device, such one or more additional elements acting in some examples as adapters. Some example cleaning machines will be described in more detail below.

Some example lumens have a lumen diameter of more than 0.5 mm and of less than 6 mm, such lumen diameter corresponding to a minimum cross section of the lumen. Lumens having non-circular cross sections may be considered as having a diameter corresponding to the cross-section concerned. In some examples, the medical device comprises different lumens having different sizes.

As illustrated in block 1002, example method 1000 comprises placing the medical device in a tank. The placing may be automated, may be as a result of human intervention, or may be as a result of both automation and human intervention. In some examples, block 1001 takes place prior to block 1002. In some examples, block 1002 takes place prior to block 1001. In some examples, blocks 1001 and 1002 are partially or wholly concomitant. In some examples, the placing of the medical device in a tank comprises placing the medical device in a specific position or orientation in the tank. In some examples, parts of the medical device may remain outside of the tank as the medical device is placed in the tank, such parts leading for example to the coupling of a lumen of the medical device to the cleaning machine. The tank should be understood as a fluid container having dimensions permitting the placing of the medical device into the tank such that fluid present in the tank may at least partially surround the medical device placed in the tank. In some examples, the tank comprises one or more of a drain, a flush and an overflow.

As illustrated in block 1003, example method 1000 comprises injecting a first volume of a first fluid from the cleaning machine, through the coupled lumen and into the tank for preliminary flushing of the lumen. It should be understood that the block 1003 in the example of FIG. 1 follows the coupling of the lumen and placing of the medical device into the tank. The injection may for example take place using an injection device such as a pump, a piston or a syringe. In some examples, the injecting of the first fluid for preliminary flushing of the lumens takes place at a pressure of more than 50 kPa and of less than 200 kPa. In some examples, the injecting of the first fluid for preliminary flushing of the lumens takes place at a pressure of more than 100 kPa and of less than 200 kPa. In some examples, the injecting of the first fluid for preliminary flushing of the lumens takes place at a pressure of more than 125 kPa and of less than 175 kPa. The first volume may in some examples be controlled to correspond to a specific quantity of first fluid, for example in order to ensure that a sufficient amount of first fluid is provided for preliminary flushing while avoiding using an excessive amount of first fluid which could otherwise result in one or more of a tank overflow, a waste of first fluid, or an insufficient or excessive dilution of second fluid in the tank in block 1008 which will be described below. In some examples, the first volume is of less than 2 L. In some examples, the first volume is of less than 1.7 L. In some examples, the first volume is of less than 1.3 L. In some examples, the first volume is of less than 1 L. In some examples, the first volume is of more than 0.5 L. Such quantities may be used to inject the first volume through a plurality of lumens concurrently, the first volume representing the total volume of first fluid injected through such plurality of lumens concurrently. The preliminary flushing may provide a substantially mechanical process, whereby at least some physiological soil or waste present in the lumen may be drained into the tank by the flow of first fluid through the lumen from the cleaning machine into the tank. While the first fluid may in some examples comprise one or more active additive such as a detergent or disinfectant, the first fluid comprises in some examples more than 95% of water by weight. In some examples, the first fluid comprises more than 99% of water by weight. The flushing is preliminary as it will be followed by further cleaning operations hereby described. In some examples, block 1003 lasts more than 5 s. In some examples, block 1003 lasts more than 10 s. In some examples, block 1003 lasts less than 60 s. In some examples, block 1003 lasts less than 30 s. In some examples, block 1003 lasts less than 20 s.

As illustrated in block 1004, example method 1000 comprises, following the injection of the first volume of the first fluid according to block 1003, injecting a second fluid from the cleaning machine, through the coupled lumen and into the tank, whereby the second fluid differs from the first fluid. One should note that in this disclosure, the wording “following” covers either blocks directly following each other, or blocks following each other but separated by an additional operation or pause which may take place between such blocks following each other. It should be understood that the second fluid follows through the lumen in block 1004 a path corresponding to the path followed by the first fluid in block 1003. The injection may for example take place using an injection device such as a pump, a piston or a syringe. In some examples, the injection takes place using a same injection device in block 1003 and in block 1004, for example in order to reduce a number of injection devices. In some examples, different injection devices are used in blocks 1003 and 1004, for example to adapt the injection to different fluid viscosities or injected fluid quantities. In some examples, the injecting of the second fluid takes place at a pressure lower than a pressure of injection of the first fluid, in some examples at least 50% lower. The volume of second fluid may in some examples be controlled to correspond to a specific quantity of second fluid, for example in order to ensure that a sufficient amount of second fluid is provided for contact with internal walls of the lumen while avoiding using an excessive amount of second fluid which could otherwise result in one or more of a tank overflow, a waste of second fluid, or an insufficient or excessive dilution of second fluid in the tank in block 1008 which will be described below. In some examples, a volume of no more than 1 L of second fluid is injected through the coupled lumen and into the tank. In some examples, a volume of no more than 0.5 L of second fluid is injected through the coupled lumen and into the tank. In some examples, a volume of no more than 0.25 L of second fluid is injected through the coupled lumen and into the tank. In some examples, a volume of no less than 0.1 L of second fluid is injected through the coupled lumen and into the tank. Such quantities may be used to inject the volume of second fluid through a plurality of lumens concurrently, the volume of second fluid representing the total volume of second fluid injected through such plurality of lumens concurrently. The second fluid differs from the first fluid in that the first and second fluid have different compositions. While a flow of the first fluid through the lumen is intended to have a substantially mechanical cleaning impact, the second fluid is intended to have an impact such as a chemical or biological impact on soil or waste by contact with such soil or waste in the lumen as will be described in block 1005. Some example compositions of the second fluid will be described below.

As illustrated in block 1005, example method 1000 comprises, following the injection of the second fluid as per block 1004, letting the second fluid sit in the lumen during a contact time period. The second fluid will thereby remain in contact in the lumen with soil or waste during such contact time period. One should understand that the second fluid may be allowed to sit in the lumen while circulating in the lumen at a substantially zero flow rate such as a flow rate of less than 10% of a flow rate attained during block 1004. In some examples, while having a substantially zero mechanical cleaning impact during block 1005, the second fluid will have a chemical or biological impact on soil or waste in the lumen during block 1005. The fact that block 1003 precedes block 1005 permits increasing such impact during block 1005 by reducing the quantity of soil or waste processed during block 1005 in the lumen compared to the quantity of soil or waste present in the lumen at an outset of block 1003, thereby enabling an increasingly focused treatment on soil or waste remaining in the lumen during block 1005. In some examples, the contact time is of more than 10 s and of less than 300 s. In some examples, the contact time is of more than 30 s and of less than 250 s. In some examples, the contact time is of more than 60 s and of less than 200 s. In some examples, the contact time is of more than 100 s and of less than 150 s. Controlling the contact time can permit ensuring sufficient treatment without introducing excessive delay.

As illustrated in block 1006, example method 1000 comprises, following the contact time period in which the second fluid sits in the lumen, injecting a second volume of the first fluid from the cleaning machine, through the coupled lumen and into the tank for rinsing the second fluid from the lumen. Such injection of a second volume of first fluid may lead to or contribute to concluding the contact time period by evacuation of the second fluid, pushing such second fluid out of the lumen and into the tank by the injection of the second volume of the first fluid. One should note that the first volume of first fluid injected in block 1003, the volume of second fluid injected in block 1004 or the second volume of first fluid injected in block 1006 each exceed in some examples an overall internal volume of the lumen. In some examples, the injecting of the first fluid for rinsing takes place at a pressure of more than 50 kPa and of less than 200 kPa. In some examples, the injecting of the first fluid for rinsing takes place at a pressure of more than 100 kPa and of less than 200 kPa. In some examples, the injecting of the first fluid for rinsing takes place at a pressure of more than 125 kPa and of less than 175 kPa. It should be understood that traces of the second fluid may or may not remain in the lumen after rinsing, such traces representing for example less than 10% of the second volume. The second volume may in some examples be controlled to correspond to a specific quantity of first fluid, for example in order to ensure that a sufficient amount of first fluid is provided for rinsing while avoiding using an excessive amount of first fluid which could otherwise result in one or more of a tank overflow, a waste of first fluid, or an insufficient or excessive dilution of second fluid in the tank in block 1008 which will be described below. In some examples, the second volume is of less than 4 L. In some examples, the second volume is of less than 3.3 L. In some examples, the second volume is of less than 2.5 L. In some examples, the second volume is of less than 1.7 L. In some examples, the second volume is of more than 1 L. Such quantities may be used to inject the second volume through a plurality of lumens concurrently, the second volume representing the total volume of second fluid injected through such plurality of lumens concurrently. 3.3 L. In some examples, the first volume of the first fluid and the second volume of the first fluid correspond to different boluses of the first fluid, permitting using “fresh” first fluid during both blocks 1003 and 1006. In some other examples, part of the first fluid of the second volume is recycled from first fluid from the first volume extracted from the tank. In some examples, rinsing as per block 1006 takes place during more than 15 s and during less than 1 min, for example during about 30 s.

As illustrated in block 1007, example method 1000 comprises, following the contact time period in which the second fluid sit into the lumen, injecting a gas through the lumen. Such injection of gas may lead to or contribute to concluding the contact time period by evacuation of the second fluid, by contributing to pushing such second fluid out of the lumen and into the tank by the injection of the second volume of the first fluid. In some examples, the gas injection is following the rinsing and is to evacuate the first fluid from the lumen. In some examples, the gas injection is to evacuate the first fluid, the second fluid, or a mixture of the first and second fluid from the lumen. It should be understood that traces of the first or second fluid may or may not remain in the lumen after evacuation, such traces representing for example less than 5% of the second volume. In some examples, block 1007 takes place after block 1006. In some examples, blocks 1006 and 1007 are partially or wholly concomitant. The injection may for example take place using an injection device such as a pump, a piston or a syringe. In some examples, the injection takes place using a same injection device in block 1007 as in block 1003 and in block 1004, for example in order to reduce a number of injection devices. In some examples, different injection devices are used in block 1007 and in blocks 1003 and 1004, for example to adapt the injection of gas compared to injection of liquids such as the first and second fluids.

As illustrated in block 1008, example method 1000 comprises, following the rinsing as per block 1006, maintaining the medical device in the tank during a soaking time, the tank containing a mixture of the first and second fluids. Such soaking may contribute to cleaning an external surface of the medical device. In some examples, the soaking time is of more than 30 s and of less than 600 s in order to impact cleaning while avoiding unduly delaying the overall cleaning process. In some examples, the soaking time is of more than 60 s and of less than 400 s. In some examples, the soaking time is of more than 90 s and of less than 200 s. In some examples, a ratio of a volume of the second fluid comprised in the tank during soaking to a total volume (first volume plus second volume) of the first fluid comprised in the tank during soaking is of less than 66%. In some examples, a ratio of a volume of the second fluid comprised in the tank during soaking to a total volume of the first fluid comprised in the tank during soaking is of less than 30%. In some examples, a ratio of a volume of the second fluid comprised in the tank during soaking to a total volume of the first fluid comprised in the tank during soaking is of less than 10%. In some examples, a ratio of a volume of the second fluid comprised in the tank during soaking to a total volume of the first fluid comprised in the tank during soaking is of more than 1.5%. A control of such a ratio permits during block 1008 a degree of treatment of external surfaces of the medical device by contact with the second fluid diluted by the first fluid, without reaching in the tank concentrations levels of the second fluid used during the contact time with inside walls of the lumen of block 1005. In some examples, an additional volume of liquid is added to the tank prior to placing the medical device in the tank or while the medical device sits in the tank, such additional liquid being added without passing through the lumen or lumens. In some examples, such additional volume of liquid is of a liquid of the same nature as the first fluid, for example water. Such addition may for example permit adjusting concentrations of components of the second fluid, for example adjusting a concentration of a surfactant of the second fluid in a concentration that is sufficient for exerting a detergent effect on the external surface of the medical device.

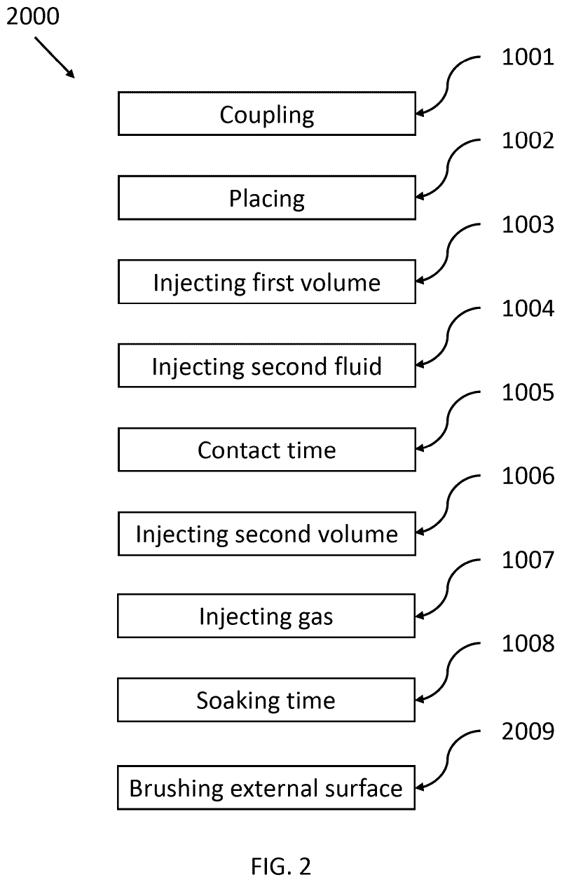

FIG. 2 represents another example method 2000 for cleaning a medical device. Method 2000 comprises block 1001-1008 as described in the context of example method 1000. Method 2000 further comprises block 2009 comprising brushing an external surface of the medical device during at least part of the soaking time. The brushing may be automated, may be as a result of human intervention, or may be as a result of both automation and human intervention.

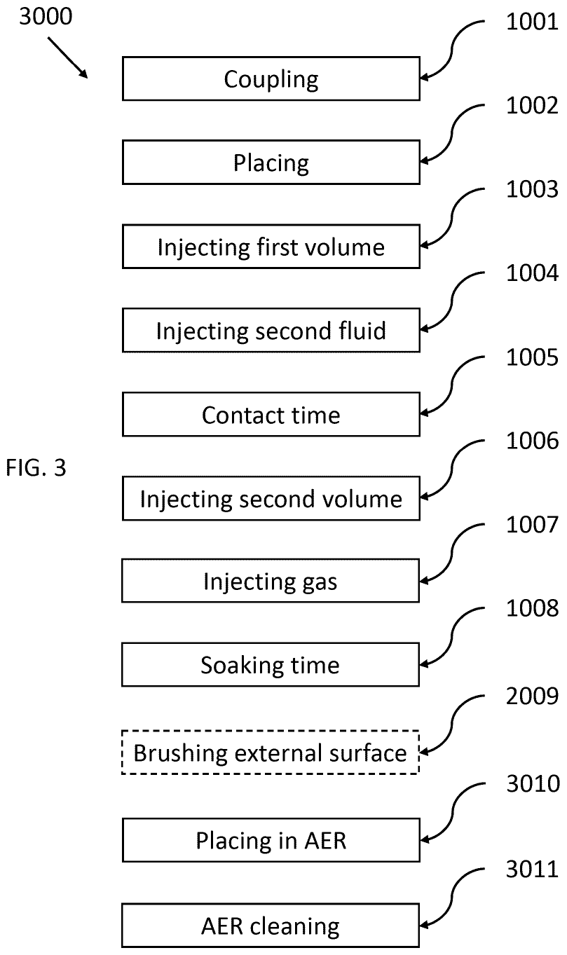

FIG. 3 represents another example method 3000 for cleaning a medical device. Method 3000 comprises block 1001-1008 as described in the context of example method 1000. Method 3000 may comprise block 2009 as described in the context of example method 2000. Method 3000 further comprises block 3010 of, following the soaking time of the lumen, placing the medical device in an automated endoscope reprocessor (“AER”). Such placing of the medical device in an AER, for example a Soluscope AER, may comprise using the same tank, or may comprise removing the medical device from the tank used in method 1000 or in method 2000. Method 3000 further comprises block 3011 of performing an AER cleaning on the medical device. Preceding the AER cleaning by a method such as method 1000 or 2000 permits casing AER cleaning. While AER cleaning may involve treating the medical device at temperatures higher than, for example, 100 degrees Celsius, in some examples the first fluid and the second fluid are at a temperature comprised between 5 degrees Celsius and 30 degrees Celsius.

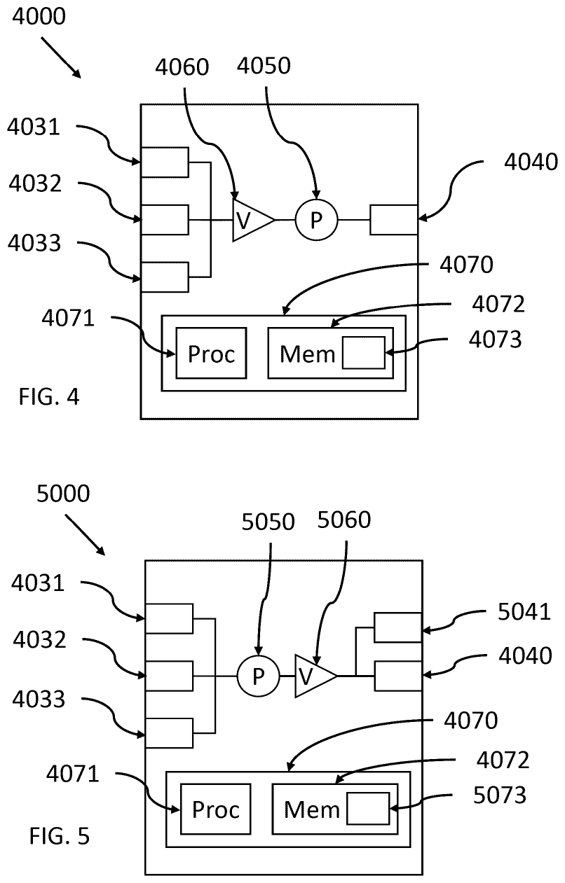

FIG. 4 represents an example cleaning machine 4000 which may be used in example methods 1000, 2000 or 3000 hereby described. Such cleaning machine 4000 comprises a first fluidic connection 4031 to a supply of a first fluid, such first fluid corresponding to the first fluid described for example in the context of block 1003. In some examples, such supply of a first fluid is a drinkable water network. In some examples, such supply of a first fluid is a combination of drinkable water and of an active component such as a detergent or disinfectant, such combination forming the first fluid. Cleaning machine 4000 further comprises a second fluidic connection 4032 to a supply of a second fluid as described in the context of block 1004. Cleaning machine 4000 further comprises a gas connection 4033 to a supply of a gas as described in the context of block 1007. In some examples, such gas is air. In some examples, the supply of gas is an air gas supply network as provided for example in a hospital. Cleaning machine 4000 further comprises a lumen connection 4040 permitting directly or indirectly coupling a lumen of a medical device to the cleaning machine as described for example in block 1002. Cleaning machine 4000 further comprises a pump 4050 and a valve 4060 to selectively supply the first fluid, the second fluid, the gas, or a mixture of these, to the lumen connection 4040 in order for the cleaning machine to operate according to blocks 1003, 1006 and 1007. Cleaning machine 4000 further comprises a control device 4070 including a processor 4071, a storage 4072 and an instruction set 4073 configured to implement any of the example methods hereby described. Instruction set 4073 may for example comprise blocks such as blocks 1003, 1004, 1006 and 1007.

Processor 4071 may comprise electronic circuits for computation managed by an operating system.

Storage 4072 should be understood as a computer readable storage, and may be any electronic, magnetic, optical or other physical storage device that stores executable instructions. The computer readable storage may be, for example, Random Access Memory (RAM), an Electrically Erasable Programmable Read Only Memory (EEPROM), a storage drive, and optical disk, and the like. As described hereby, the computer readable storage may be encoded with executable instructions implementing the printer controllers hereby described. Storage or memory may include any electronic, magnetic, optical or other physical storage device that stores executable instructions as described hereby.

Storage 4072 may be a non-transitory machine-readable storage medium encoded with instructions executable by a processor such as processor 4071, the machine-readable storage medium comprising instructions to operate a cleaning machine according to any of the example methods hereby described.

In some examples, the control device or controller 4070 further comprises a network interface to receive data, wirelessly or by wire. In other examples, data may be provided locally, for example through a connector such as a USB connector. In some examples the controller 4070 further comprises a reader to read an identification tag which may be electromagnetically interrogated. A combination of a network interface and of such a reader may facilitate medical device tracking.

FIG. 5 represents an example cleaning machine 5000 which may be used in example methods 1000, 2000 or 3000 hereby described. Such cleaning machine 5000 comprises elements 4031, 4032, 4033, 4040, 4070, 4071 and 4072 as described in the context of FIG. 4. Cleaning machine 5000 comprises an additional lumen connection 5041. Indeed, in some examples, the lumen connection 4040 pertains to a coupling block of the cleaning machine, the coupling block comprising a plurality of lumen connections such as lumen connections 4040 and 5041. Such example cleaning machine comprises a pump 5050 and a valve 5060 configured to selectively supply the first fluid, the second fluid, the gas, or a mixture of these, to the respective lumen connections 4040 and 5041. An instruction set 5073 similar to instruction set 4073 may be adapted to operate using a plurality of lumen connections. In some examples, the plurality of lumens pertain to a same medical device. In some examples, the plurality of lumens pertain to different medical devices.

In some examples whereby the medical device comprises a plurality of lumens and whereby the cleaning machine comprises a plurality of corresponding lumen connections, each lumen is submitted to the preliminary flushing, to the second fluid injection, to the contact time period, to the rinsing and to the gas injection. Instruction set 5073, for example, may be adapted to operate the cleaning machine accordingly. In some examples, each lumen is fluidically coupled to a respective lumen connection of the cleaning machine, permitting selectively operating each lumen independently. In some examples, a single lumen connection of the cleaning machine is activated at a given time, permitting for example operating with a pump having a limited nominal power. In some examples, a group of the lumen connections are activated in parallel at a given time, permitting for example parallel processing of such lumens to gain processing time. In some examples, the group of lumen connections corresponds to lumen connections having similar sizes, for example whereby each lumen of the group having a diameter within 10% of an average diameter of the lumens of the group, whereby such similar sizes ensure a similar behavior during cleaning, avoiding that a lumen having a significantly smaller diameter than another experiment a limited fluid flow by pressure loss. In some examples, the group of lumen connections corresponds to lumen connections having sizes smaller, for example at least 50% smaller in minimum cross section, than a size of at least some other lumen connections of the plurality of lumens. In some examples, the activation in parallel corresponds to the rinsing of the lumens.

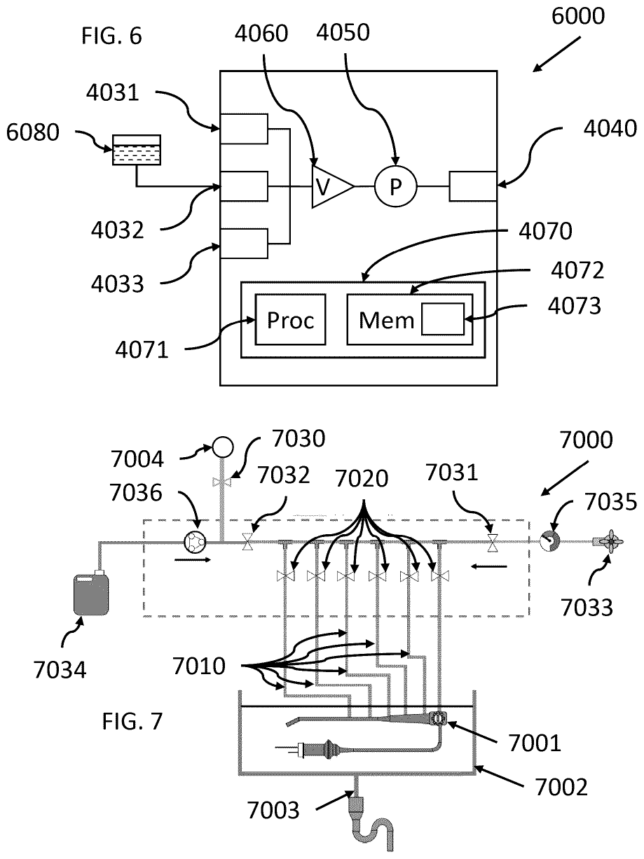

FIG. 6 represents an example cleaning machine 6000 which may be used in example methods 1000, 2000 or 3000 hereby described. Such cleaning machine 6000 comprises elements 4031, 4032, 4033, 4040, 4050, 5060, 4070, 4071 and 4072 as described in the context of FIG. 4. Cleaning machine 6000 further comprises a container 6080 for the second fluid and a reserve of second fluid in the container, whereby the second fluid, as described for example in the context of block 1004, has a composition as per any of the example compositions hereby described.

FIG. 7 represents an example cleaning machine 7000 for cleaning a medical device 7001, whereby the medical device 7001, in this example an endoscope, comprises 6 different lumens. Each lumen is connected to a respective lumen connection, the cleaning machine 7000 thereby comprising lumen connections 7010. In this example, each lumen connection comprises a flexible connection tube. Each lumen connection is in this example connected to a respective valve 7020. Each valve 7020 is in turn connected to a first fluid valve 7031 and to a second fluid valve 7032. The valve 7031 is in turn connected to a drinking water (drinking water being the first fluid in this case) supply network 7033 acting as first fluidic connection, such water supply network 7033 providing water supply at a water supply pressure controlled by a water pressure gauge 7035. The valve 7032 is in turn connected to a second fluid container 7034, such container the second fluid to the valve 7032 through a pump 7036. In this example the medical device 7001 is placed in a tank 7002 provided with a drain 7003 to empty the tank. The gas connection to operate block 1007 is provided at 7004, for example a connection to a compressed air network or compressed air container, through a valve 7030.

One should note that different valve types may be used in a cleaning machine according to this disclosure, whereby a valve should be understood as a device which regulates flow of one or more fluids by opening, closing or partially obstructing one or more fluid passages.

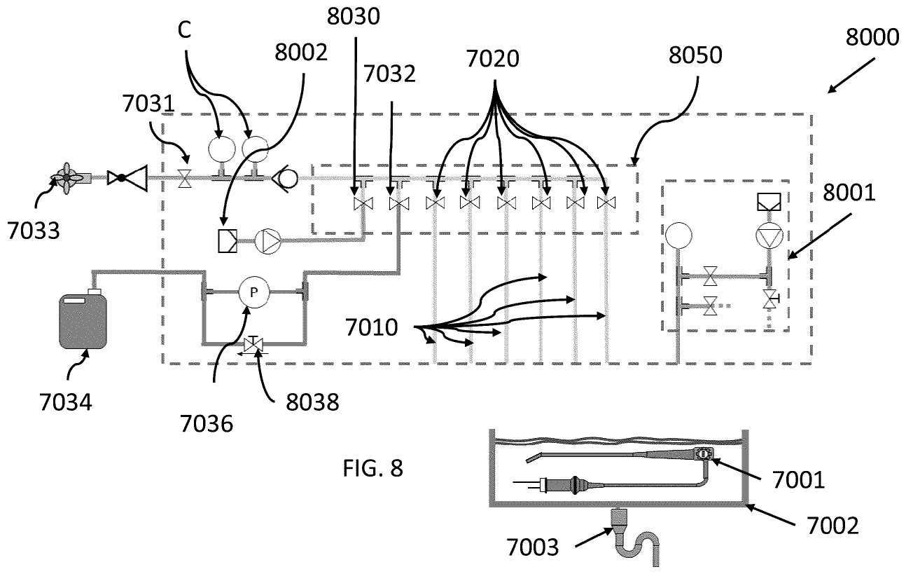

FIG. 8 represents an example cleaning machine 8000 comprising elements 7001, 7002, 7003, 7010, 7020, 7031, 7032, 7033, 7034 and 7036 as described in the context of FIG. 7. Cleaning machine 8000 further comprises a leak test block 8001 which may be used to check lumen connections for leaks, and an air purge circuit 8002 as gas connection to supply the gas according to block 1007 through a gas valve 8030. In this illustration, the connections to the medical device are not represented. In this example, additional connections C are provided downstream from the water supply network to for example introduce a detergent or a disinfectant into the water into the first fluid. In this example, a return valve 8038 is provided in parallel to pump 7036 for the second fluid. In this example, valves 7020, 7032 and 8030 are comprised in a mixing block 8050.

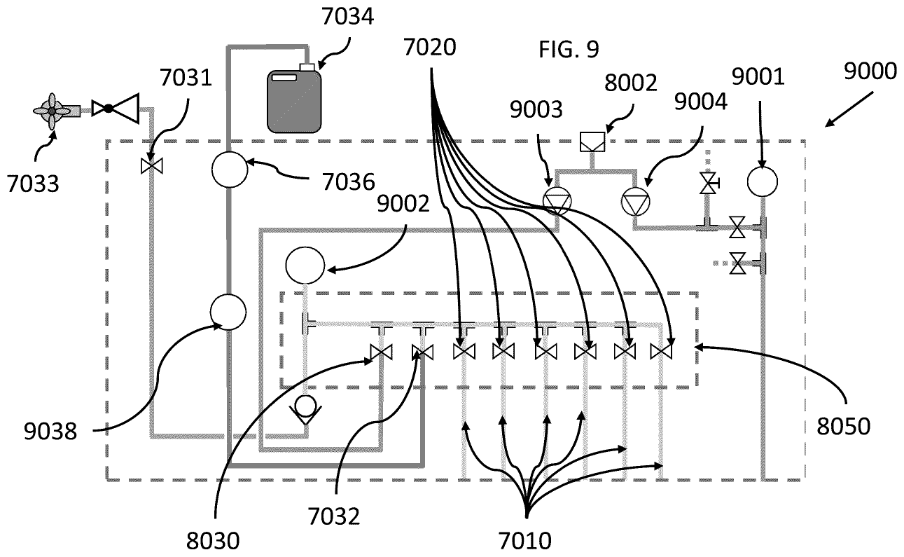

FIG. 9 represents an example cleaning machine 9000 comprising elements 7010, 7020, 7031, 7032, 7033, 7034, 7036, 8030, 8050 and 8002 as described in the context of FIG. 7 or 8. Cleaning machine 9000 further comprises a sensor 9038 located between the pump 7036 and the valve 7032 for the second fluid and permitting monitoring characteristics of the second fluid. In this illustration, the connections to the medical device, and the medical device, are not represented. In this example, a leak sensor 9001 is provided as part of the gas connection. In this example, a general fluid pressure sensor 9002 is provided as part of the fluid connections circuit. A pump purge 9003 and a pump leak sensor 9004 are also provided downstream from the gas connection 8002, the pump purge being located upstream from the gas valve 8030.

In some examples, an example cleaning machine operating according to hereby described example methods using hereby described example compositions permit partially or completely replacing a brushing step of lumens of medical devices such as endoscopes, participating to an endoscope reprocessing cycle with an automated, replicable, faster process, elimination or reduction of human errors and improved security for patients and users. In some other processes, the brushing step of lumens of endoscope reprocessing cycles are done manually by healthcare professionals. This manual task can lead to debris and bioburden which can accumulate and biofilms may develop inside channels (i.e. inside lumens) over time through use, which may pose an infection risk. In such other processes, healthcare professionals may also be affected by elbow pain or repetitive strain injury, time waste and waste management due to brushing of lumens as a manual task. Example cleaning machines of methods according to this disclosure can allow such issues to disappear or be reduced and non-lumen-brushing process may be to skip a manual lumen brushing step from an endoscope reprocessing cycle. This is particularly efficient in narrow lumens such as lumens having a minimum diameter of less than 3 mm, less than 2 mm or even less than 1.5 mm which are difficult to brush or cannot be brushed. The replacement of manual endoscope brushing with an automated process as per examples hereby described can be made with no additional time, avoiding or reducing human errors, and simplifying the process.

In some examples, the cleaning machine is a tabletop or wall maintained electrical equipment. In some examples, the cleaning machine is connected to an electrical power grid, a drinking water system of a hospital which may for example provide the first fluid and a computer network, for example for medical device tracking purposes. In some examples, the cleaning machine is by weight and scale wearable by a single human. In some examples, the cleaning machine comprises an integrated touchscreen, a connection system to Soluscope endoscope connectors and a system to identify a user, a product, or an endoscope. In some examples, the cleaning machine is compatible with Soluscope software solutions. In some examples, the cleaning machine integrates electric, electronic, pneumatic, hydraulic and mechanical components to perform. Example of such components are: pumps, in particular diaphragm pumps, industrial automates, solenoid valves, for example solenoid valves to allow for selective lumen treatment, air compressors, sheet metal structures, Human Machine Interfaces (HMI) permitting controlling pumps, valves, gas and fluids hereby described, and other components. Components exposed to chemical compositions, in particular to the second fluid, should be made from a compatible material. Example tubing material for lumen connections comprises PTFE (Polytetrafluoroethylene). In some examples, a lumen connection is a tube having a length of more than 50 cm, more than 1 m, or more than 1.5 m. In some examples, a lumen connection is a tube having a length of about 2 m.

In another aspect, the present disclosure provides a composition for cleaning a medical device.

More particularly, it is hereby provided a composition for cleaning a medical device, such as in particular an endoscope, that may be used in the methods for cleaning of medical device described herein. Such composition is preferably intended to be used as a second fluid in the example methods for cleaning a medical device described herein. Such composition may be operated with the cleaning device described herein.

Such composition may be operated with the cleaning device or implemented in the method for cleaning a medical device as described herein and thereby contributes to the cleaning of such medical device.

As detailed above, the cleaning should be understood as removing at least some soil and/or waste.

In some embodiments, the composition used as a second fluid in the method for cleaning a medical device as described herein contribute to the cleaning of the lumen of medical device.

It some embodiments, the composition used as a second fluid in the method for cleaning a medical device as described herein may also contribute to the cleaning of the external surface of the medical device.

In some embodiments, the composition contributes both to the cleaning of the lumen of the medical device and to the cleaning of the external surface of the medical device.

Soils that dirty the medical device are usually proteinaceous in nature. Test soils mimic soiling from medical procedures. Such test soils e.g. those according to ISO 15883-5:2005 create worst-case soiling on reusable medical devices such as endoscopes. Examples of test soils according to ISO 15883-5:2005 are defined in the Table below:

| Code | Soil composition |
|---|---|
| Austria | Nigrosin, wheatflour, egg, E. Facium. |
| Germany | heparinized blood previously added with protamine |
| | sulphate |
| Biofilm test soil | Biofilm formed by Pseudomonas aeruginosa |

The main chemical substances to be assayed (i.e. analytes) in ISO 15883-5:2005 are protein and TOC (Total Organic Carbon). According to ISO 15883-5:2005, “action level” refers to value from monitoring that necessitates immediate intervention. This corresponds to a maximum value of analyte not to be exceeded. According to ISO 15883-5:2005, “alert level” refers to value from monitoring that value from monitoring providing early warning of deviation from specified conditions. This corresponds to the target value of analyte.

According to ISO 15883-5:2005, the action and alert levels for protein and TOC in connection with German soil and Austrian soil and biofilm soil are as in the Table below:

| Analytes | Alert level | Action level | |
|---|---|---|---|
| Proteins | ≥3 μg/cm² | ≥6.4 | μg/cm² |
| Total Organic Carbon | ≥6 μg/cm² | ≥12 | μg/cm² |

The inventors have found that a composition containing hydrogen peroxide as described herein, when used as a second fluid in the example methods for cleaning a medical device as described herein, are able to remove a wide range of test soils according to ISO 15883-5:2005 test soils (“Test soils and methods for demonstrating cleaning efficacy”), including test soils known to be particularly challenging to remove such as German soil.

In particular, such composition allows to clean a medical device such as an endoscope, in particular its lumen, with contact time periods of e.g. 2 minutes and at ambient temperatures e.g. 20° C.

In particular, when the method for cleaning is applied to the lumen of a medical device soiled with a German soil, an Austrian soil, or a biofilm soil, using the composition as described herein as a second fluid according to examples hereby described, the cleaned medical device has a protein and TOC level equal or below the alert level as set in ISO 15883-5:2005.

The disclosure provides a composition for cleaning of a medical device, wherein the composition is in the form an aqueous solution comprising hydrogen peroxide and optionally one or more of a surfactant, a sequestrant, and an adjusting pH agent, and wherein the composition has a pH of less than 8.

### <Hydrogen Peroxide>

The composition comprises hydrogen peroxide.

Without wanting to be bound by any theory, the inventors believe that the presence of hydrogen peroxide in the composition is crucial to obtain a cleaning effect on various test soils, including the most challenging ones such as German soil.

The content of hydrogen peroxide is not particularly limited. It is preferable to have a minimum content of hydrogen peroxide. That minimal content of hydrogen peroxide may depend on other components of the composition, in particular on the presence of a sequestrant. More particularly, without wanting to be bound by a theory, the inventors believe that the presence of a sequestrant may potentiate the cleaning effect of the hydrogen peroxide, as shown in the experimental part below.

The content of the hydrogen peroxide with respect to the total mass of the composition is preferably of 5% or more, more preferably of 6% or more, more preferably of 7% or more, more preferably of 8% or more, % by weight of the composition.

The upper limit of the content of hydrogen peroxide with respect to the total weight of the composition is preferably 30% or less, more preferably 20% or less, even more preferably 15% or less, % by weight of the composition.

In some embodiments, the content of hydrogen peroxide is preferably between 5 and 30%, more preferably 5 and 20%, more preferably between 5 and 15%, % by weight of the composition.

In some embodiments, the content of hydrogen peroxide is preferably between 7 and 30%, more preferably 7 and 20%, more preferably between 8 and 15%, % by weight of the composition.

In some embodiments, the content of hydrogen peroxide is preferably between 8 and 20%, more preferably 8 and 15%, more preferably between 9 and 11%, % by weight of the composition.

Hydrogen peroxide is an oxidizing agent that rapidly destabilizes agent in alkaline conditions. To provide a composition having a minimum of stability over time, the pH of the composition must be equal or less than 8.

In some examples, the pH of the composition is comprised between 2 and 8, between 2 and 7, or between 2 and 6. In some examples the pH is comprised between 3 and 8, between 3 and 7 or between 3 and 6. In some examples, the pH is comprised between 4 and 8, between 4 and 7 or between 5 and 7.

In order to provide a composition with a pH as above, the composition may comprise a pH adjusting agent as described herein.

### <Surfactant>

The composition may comprise a surfactant.

The surfactant shall be stable in the presence of a hydrogen peroxide, which is a strong oxidant. This limits the nature of the surfactant that can be used in the composition described herein.

The surfactant is preferably selected from an alkyl dimethyl amine oxide, a sulfate, an ether sulfate, a phosphate ester, and mixtures thereof.

Advantageously, the surfactant is an alkyl dimethyl amine oxide.

In particular, the above alkyl dimethyl amine oxide may comprise a C8-18 alkyl moiety (i.e. C8-18 alkyl dimethyl amine oxide), preferably a C12-C14 alkyl moiety (i.e. C12-C14 alkyl dimethyl amine oxide).

In some embodiments, following the rinsing, the mixture of the first and second fluids contained in the tank comprises the surfactant in a concentration that is sufficient for exerting a detergent effect on the external surface of the medical device. Without wanting to be bound by any theory, the inventors believe that the detergent effect on the external surface of the medical device in the tank may also be linked to a synergy between the surfactant and the sequestrant.

The content of surfactant is preferably between 1 and 10%, more preferably between 1 and 7%, more preferably between 2 and 6%, % by weight of the composition.

### <Sequestrant>

The composition may comprise a sequestrant.

The presence of a sequestrant in the composition is advantageous. Without wanting to be bound by a theory, the inventors believe that the sequestrant essentially allows the composition to exert an effect on biofilms.

In some embodiments, the sequestrant is selected from MGDA (methylglycine N,N-diacetic acid), IDS (iminodisuccinate), GLDA (glutamate diacetate), EDTA (ethylenediaminetetraacetic acid, NTA (nitrilotriacetic acid), and mixtures thereof.

The sequestrant is for example MGDA.

The content of sequestrant is not particularly limited. The content of sequestrant is preferably between 1 and 10%, more preferably between 1 and 7%, more preferably between 2 and 6%, % by weight of the composition.

### <pH Adjusting Agent>

The composition may comprise a pH adjusting agent selected from a phosphonate or an acid.

In some embodiments, the pH adjusting agent is a phosphonate. Indeed, phosphonate allows to lower the pH and can at the same time exert disinfecting properties.

The phosphonate may for example be selected from HEDP (1-hydroxyethylidene-1,1-diphosphonic acid), ATMP (amino trimethylene phosphonic acid), DTPMP (diethylenetriamine penta (methylenephosphonic acid)), PBTC (2-Phosphonobutane 1,2,4-tricarboxylic acid), BHMTPMP (bis (hexamethylene) triaminepentakis (methylene phosphonic acid)), and mixtures thereof.

The phosphonate is preferably HEDP. Without wanting to be bound by a theory, the inventors have shown that HEDP have some stabilizing effect on hydrogen peroxide in the composition described herein.

The content of pH adjusting agent may be adjusted depending on pH desired for the composition.

When the pH adjusting agent is a phosphonate, its content is preferably between 1 and 10%, more preferably between 1 and 7%, more preferably between 1 and 4%, % by weight of the composition.

### <Viscosity>

The composition is an aqueous solution in liquid form. More particularly, the composition preferably has a low viscosity allowing it to pass through a wide variety of lumen diameters preferably including lumen having diameters inferior or equal to 1 mm. Such low viscosity also eases the rinsing of the composition within the lumen.

In some embodiments, the composition has a viscosity of less than 100 mPa/s. In some embodiments, the composition has a viscosity of at least 5 mPa/s and of less than 100 mPa/s. In some embodiment, the composition has a viscosity between 5 and 100 mPa/s, preferably between 5 and 80 mPa/s, preferably between 5 and 60 mPa/s, preferably between 5 and 40 mPa/s, preferably between 5 and 20 mPa/s.

In some embodiments, the composition has a viscosity of at least 6 mPa/s and of less than 50 mPa/s.

The viscosity of the composition as described herein is preferably determined at 20° C. using a Brookfield DVIII Ultra rheometer at speed rate between 75 and 250 rpm.

The viscosity of the composition may be adjusted by the addition of a fluidifying agent.

### <Fluidifying Agent>

The composition may further comprise a fluidifying agent.

The fluidifying agent is used to adjust the viscosity of the composition.

In some embodiments, the fluidifying agent is selected from an alcohol, a propylene glycol ether, and mixtures thereof.

The fluidifying agent is for example 1,2 propane-glycol.

### <Water>

The composition is an aqueous solution containing water.

The nature of water is not particularly limited, and examples thereof include demineralized water, distilled water, deionized water, and pure water.

The content of water in the composition is not particularly limited. The content of water is preferably 20% or more, more preferably 30% or more. The upper limit of the content of water is less than 100%, preferably 90% or less, and more preferably 80% or less, % in weight of the composition.

### <Other Components>

The composition may further comprise other agents such as a dispersion or solubility enhancer, a perfume or a dye.

### <Dye>

The composition may further comprise a dye.

Any kind of dye may be used. The dye is preferably a blue dye.

The dye may for example be an organometallic dye based on copper, chromium or nickel.

The dye is for example CAS 13-30-38-7 known under its commercial name Direct Blue 86.

The content of dye in the composition is not particularly limited. The content of dye is preferably of at least 0.0005%, % in weight of the composition. It can for example be comprised between 0.0005 and 0.1, preferably between 0.0005 and 0.5%, even more preferably between 0.0010 and 0.0030%, and more preferably of about 0.0025%, % in weight of the composition.

### EMBODIMENTS

In some embodiment, the composition described herein comprises hydrogen peroxide and a sequestrant preferably selected from MGDA, IDS, GLDA, EDTA, NTA, and mixtures thereof.

In some embodiment, the composition described herein comprises hydrogen peroxide and a surfactant preferably selected from an alkyl dimethyl amine oxide, a sulfate, an ether sulfate, a phosphate ester, and mixtures thereof.

In some embodiment, the composition described herein comprises hydrogen peroxide; a sequestrant preferably selected from MGDA, IDS, GLDA, EDTA, NTA and mixtures thereof; and a surfactant preferably selected from an alkyl dimethyl amine oxide, a sulfate, an ether sulfate, a phosphate ester, and mixtures thereof.

In some embodiment, the composition described herein comprises hydrogen peroxide; a sequestrant preferably selected from MGDA, IDS, GLDA, EDTA, NTA and mixtures thereof; a surfactant preferably selected from an alkyl dimethyl amine oxide, a sulfate, an ether sulfate, a phosphate ester and mixtures thereof, and as a pH adjusting agent a phosphonate preferably selected from HEDP, ATMP, DTPMP, PBTC, BHMTPMP and mixtures thereof.

In some embodiment, the composition described comprises:
at least 5% preferably between 8% and 20%, of hydrogen peroxide,
between 1% and 10% of sequestrant, preferably between 4 and 10% of sequestrant,
optionally, one or more of a surfactant, an adjusting pH agent, a fluidifying agent, a dye, % by weight of the composition,
   preferably wherein:
the surfactant is selected from an alkyl dimethyl amine oxide, a sulfate, an ether sulfate, and a phosphate ester and mixtures thereof and mixtures thereof;
the sequestrant is selected from MGDA, IDS, GLDA, EDTA, NTA and mixtures thereof;
the adjusting pH agent is a phosphonate preferably selected from HEDP, ATMP, DTPMP, PBTC, BHMTPMP and mixtures thereof; and/or
the fluidifying agent is selected from an alcohol, a propylene glycol ether;
   optionally wherein the composition further comprises water qs 100%, the sum of the components making 100%.

In some embodiment, the composition described herein comprises:
at least 5%, preferably between 8% and 20% of hydrogen peroxide,
between 1% and 10% of surfactant,
between 1% and 10% of sequestrant,
between 1% and 10% of adjusting pH agent,
between 0% and 20% of fluidifying agent,
between 0% and 0.1% of dye,
   % by weight of the composition,
   preferably wherein:
the surfactant is selected from an alkyl dimethyl amine oxide, a sulfate, an ether sulfate, and a phosphate ester and mixtures thereof and mixtures thereof;
the sequestrant is selected from MGDA, IDS, GLDA, EDTA, NTA and mixtures thereof;
the adjusting pH agent is a phosphonate preferably selected from HEDP, ATMP, DTPMP, PBTC, BHMTPMP and mixtures thereof; and/or
the fluidifying agent is selected from an alcohol, a propylene glycol ether;
   optionally wherein the composition further comprises water qs 100%, the sum of the components making 100%.

In some embodiment, the composition described herein comprises:
between 8% and 20%, preferably between 8 and 15% of hydrogen peroxide,
between 1% and 4% of surfactant,
between 2% and 6% of sequestrant,
between 2% and 10% of phosphonate,
between 0% and 20% of fluidifying agent,
between 0% and 0.1% of dye,
   % by weight of the composition;
   preferably wherein:
the surfactant is selected from an alkyl dimethyl amine oxide, a sulfate, an ether sulfate, and a phosphate ester and mixtures thereof and mixtures thereof;
the sequestrant is selected from MGDA, IDS, GLDA, EDTA, NTA and mixtures thereof;
the phosphonate is selected from HEDP, ATMP, DTPMP, PBTC, BHMTPMP and mixtures thereof; and/or
the fluidifying agent is selected from an alcohol, a propylene glycol ether;
   optionally wherein the composition further comprises water qs 100%, the sum of the components making 100%.

In some embodiments, the composition comprises:
about 10.5% of hydrogen peroxide,
about 2.4% of surfactant, preferably of HEDP,
about 4% of sequestrant, preferably MGDA,
about 6% of an alkyl dimethyl amine oxide, preferably an C12-C14 alkyl dimethyl amine oxide,
about 10% of a fluidifying agent, preferably propane 1,2 diol,
about 0.0025% of dye,
   % by weight of the composition,
   optionally wherein the composition further comprises water qs 100%, the sum of the components making 100%.

According to the present disclosure, the term “about” herein when referring to an amount, a temporal duration, and the like, is meant to encompass variations of +/−5% or less, more preferably +/−2%, and more preferably +/−1% or less of and from the specified value, insofar such variations are appropriate to perform in the disclosed invention.

### <Composition Manufacturing Method>

The method for manufacturing the composition is not particularly limited. Any manufacturing method can be used. Examples of manufacturing method include a method comprising mixing together predetermined amounts of water, a hydrogen peroxide containing solution and one or more components e.g. surfactant or sequestrant containing raw material.

The pH of the composition must be of less than 8, and more preferably less than 7.

In particular, it can be noted that the raw material of some components such as MGDA may contain sodium hydroxide and thus have a certain degree of alkalinity.

In order to adjust the pH, the composition may contain a pH adjusting agent as described herein.

<Composition container>

The composition may be stored in a container, for example container 6080 or container 7034, and kept as it is until use.

In the present disclosure, the container and the composition stored in the container are collectively referred to as “composition container”. The stored composition is used after being taken out of the composition container. It is also preferable that the composition be transported as a composition container and provided from the manufacturer to the user, from the storage place to the place of use.

It is also preferable that the container have a degassing mechanism for adjusting the pressure (internal pressure) in the container due to the presence of hydrogen peroxide. The degassing mechanism is, for example, a mechanism which releases the generated gas from the inside of the container to the outside in a case where gas is generated from the composition by the increase in temperature of the composition in the container during storage of the composition container and/or by the decomposition of some components of the composition, in particular oxygen peroxide, so that the internal pressure is kept within a certain range without excessively increasing.

Examples of the degassing mechanism include a check valve.

As a cap of the container, it is also preferable to adopt a degassing cap comprising a degassing mechanism, so that the container includes a degassing mechanism.

That is, it is also preferable that the container of the composition container have a degassing cap comprising a degassing mechanism that adjusts the internal pressure of the container.

In view of ease of handling of the composition, it is preferable that the composition be provided from the manufacturer to the user, from the storage place to the place of use in the form of such a composition container.

Examples of the degassing cap include a cap provided with a valve that releases the internal gas of the container to the outside in a case where pressure (internal pressure) over a certain level is applied to the cap.

The present disclosure also provides the use of the composition or composition container as described above for the cleaning of a medical device.

The present disclosure is further illustrated by the following examples.

### EXAMPLES

### Example 1

To ensure the efficiency of the process trials was carried on following the methodologies describe below:

### Material and Methods

### 1/Activities Against Mono Bacterial Biofilm:

The test realized permitted to estimate the cleaning power against a mono bacterial biofilm artificially developed in a PTFE tube according to the referenced method described in the Standard: ISO 15883-5:2005.

The lumen of a PTFE tube was filled with a Pseudomonas aeruginosa CIP A 22 suspension at 108 CFU/ml.

The lumen is then supplied with a specific culture media during 96 hours at 30° C.

The cleaning activities of the process was tested on the biofilm obtained in the PTFE tube lumen.

At the end of the test, different sections of the tube were plunged in a recovery solution with sand.

Decimal dilutions of the eluate obtained are used to determine the residual protein the inside PTFE tube.

The tests were made on different diameter of PTFE channel: 2 and 4 mm.

The requirements for cleaning efficacy tested were as defined in ISO 15883-5:2005. The maximum acceptable level of protein on a cleaned device shall be lower than the action level for each sample, as detailed in the Table below:

| Analytes | Alert level | Action level |
|---|---|---|
| Proteins | ≥3 μg/cm² | ≥6.4 μg/cm² |

### 2/Activities Against German Soil:

PTFE tubes 2 meters long and with different diameters (2 mm and 5 mm) were soiled with a mixture of heparinized blood previously added with protamine sulphate.

The test soil was prepared by adding 150 μL of protamine to 10 mL of heparinized blood. The test soil has to be used immediately before its coagulation.

This test soil is clinically relevant and particularly challenging as it contains blood which can adhere to endoscope channels if dried. It was first proposed for type testing of the cleaning efficacy on surgical instruments and endoscope (ISO 15883-5:2005) and is considered as a reasonable indicator for testing cleaning efficacy on endoscopes.

The tubes soiled with 10 ml of coagulated blood were left to dry at room temperature for 1 hour before carrying out the performance test.

At the end of the test, samples were taken from the tubes to determine the amount of residual protein and determine the TOC (total organic carbon):

### Proteins Dosage Kit:

Name: MicroBC Assay Protein Quantitation Kit
Manufacturer: Interchim

### TOC Measurement Device:

Name: COT-mètre Thermique TOC-L
Manufacturer: SHIMADZU

Similar tests were carried out on an endoscope surrogate: surrogate Fuji G5/8 composed of 4 channels of 3 meters having different diameters: Two 2 mm diameter channels, one 4.8 mm diameter channel, one 1.2 mm diameter channel.

The channels are marked with colored rings to ease their identification:
channels 2 mm diameter: one channel marked in green and the other one is marked in blue
the channel 4.8 mm diameter is marked in yellow.

For these tests, all the surrogate channels were relieved and treated following the process below:
Flushing of each channel with tap water at 1000 mPa for 15 seconds,
Filling of each channel with the chemistry (i.e. composition containing hydrogen peroxide),
Contact time: 2 minutes,
Flushing of each channel with tap water at 1000 mPa during 30 seconds

Test requirements for cleaning efficacy tests as defined in ISO 15883-5:2005. The maximum acceptable level of protein on a cleaned device shall be lower than the action level for each sample, as described below.

| Analytes | Alert level | Action level | |
|---|---|---|---|
| Proteins | ≥3 μg/cm² | ≥6.4 | μg/cm² |
| Total Organic Carbon | ≥6 μg/cm² | ≥12 | μg/cm² |

### 3/Activities Against Austrian Soil:

PTFE tubes 2 meters long and with different diameters were soiled with an artificial soil made up of Nigrosine, wheat flour, egg.

The tubes were soiled with 10 ml of Austrian soil and left to dry at room temperature for 1 hour before carrying out the performance test.

At the end of the test, samples were taken from the tubes to determine the amount of residual protein and determine the TOC:

### Proteins Dosage Kit:

Name: MicroBC Assay Protein Quantitation Kit
Manufacturer: Interchim

### TOC Measurement Device:

Name: COT-mètre Thermique TOC-L
Manufacturer: SHIMADZU

Similar tests were carried out on a surrogate: surrogate Fuji G5/8 (5 mm diameter). Composed of 4 channels: Two 2 mm diameter channels, one 4.8 mm diameter channel, one 1.2 mm diameter channel.

For the tests, all the surrogate channels were relieved and treated following the process below:
Flushing each channel with tap water at 1000 mPa for 15 seconds,
Filling each channel with the chemistry,
Contact time: 2 minutes,
Flushing of each channel with tap water at 1000 mPa for 30 seconds.

For these tests, all the surrogate channels were relieved and treated according to the method of the invention.

Test requirements for cleaning efficacy tests as defined in ISO 15883-5:2005. The maximum acceptable level of protein on a cleaned device shall be lower than the action level for each sample, as detailed in the Table below:

| Analytes | Alert level | Action level | |
|---|---|---|---|
| Proteins | ≥3 μg/cm² | ≥6.4 | μg/cm² |
| Total Organic Carbon | ≥6 μg/cm² | ≥12 | μg/cm² |

### Results

1^{st} Set of Experiments: Treatment without Chemistry at 1 Pa

PTFE tubes are treated according to the process:
first flush with tap water for 15 seconds at 1 Pa
second flush with tap water for 30 seconds at 1 Pa.

The results are presented in the Table below:

| PTFE channel | Soil | Results COT: μg/cm² |
|---|---|---|
| 5 mm diameter | Austrian | 0.16 |
| 5 mm diameter | Austrian | 0.17 |
| 5 mm diameter | Austrian | 0.16 |
| 5 mm diameter | Austrian | 0.17 |
| 5 mm diameter | German | 13.01 |
| 5 mm diameter | German | 11.85 |
| 5 mm diameter | German | 9.41 |
| 5 mm diameter | German | 9.34 |
| 5 mm diameter | German | 21.99 |
| 5 mm diameter | German | 20.78 |
| 5 mm diameter | German | 22.87 |
| 5 mm diameter | German | 23.42 |
| PTFE channel | Soil | Results Protein: μg/cm² |
| 5 mm diameter | Biofilm | 12.34 |
| 5 mm diameter | Biofilm | 11.92 |

The results above show that the succession of two flushes at 1 Pa of pressure with tap water are sufficient to reach the desired values for Austrian soil. However, this is not sufficient to obtain the desired values for German soil and biofilm.

2^{nd} Set of Experiments: Treatment without Chemistry at 0.2 Pa, 0.5 Pa, 1 Pa, 1.5 Pa

Below are some tests with different water pressure values during flush:

Tests carried out on German soiling in PTFE tubes with a diameter of 5 mm according to the following process:
First flush with tap water for 15 seconds,
Second flush with tap water for 30 seconds.

The results are presented in the Table below:

| | |
|---|---|
| Pressure (Pa) | Protein (μg/cm²) |

| | | |
|---|---|---|
| 0.2 | Pa | 360 |
| 0.6 | Pa | 275 |
| 1 | Pa | 41 |
| 1.5 | Pa | 18 |

The results show:
the influence of flush pressure: at a pressure greater than or equal to 1 Pa, the quantity of residual protein is much lower compared to lower pressures,
that the succession of two flushes at a pressure as high as 1.5 Pa with tap water is still not sufficient to reach the desired values for German soil.
   3^{rd} Set of Experiments: Treatment with Chemistry (Formula #02) in PTFE Channels

In the tables below some examples of results obtained with a composition of formula #02:
10.5% of hydrogen peroxide,
6% of surfactant (C12-14 alkyl dimethyl amine oxide),
4% of sequestrant (MGDA),
2.4% of phosphonate (HEDP),
10% of fluidifying agent (Propane-1,2-diol),
water qs 100%.

Results on PTFE channel (diameter 2 mm or 5 mm):

German soil: COT

| 2 meters PTFE | | | Results COT: |
|---|---|---|---|
| channel | Soil | Formula | μg/cm² |
| 5 mm diameter | German | #02 | 1.82 |
| 5 mm diameter | German | #02 | 1.82 |
| 5 mm diameter | German | #02 | 0.30 |
| 5 mm diameter | German | #02 | 0.31 |
| 5 mm diameter | German | #02 | 1.82 |
| 5 mm diameter | German | #02 | 1.84 |
| 5 mm diameter | German | #02 | 1.39 |
| 5 mm diameter | German | #02 | 1.31 |
| 5 mm diameter | German | #02 | 2.22 |
| 5 mm diameter | German | #02 | 2.25 |

German soil: protein

| 2 meters PTFE | | | Results Protein |
|---|---|---|---|
| channel | Soil | Formula | μg/cm² |
| 5 mm diameter | German | #02 | 0.303 |
| 5 mm diameter | German | #02 | 0.229 |
| 5 mm diameter | German | #02 | 0.13 |
| 5 mm diameter | German | #02 | 0.3 |
| 5 mm diameter | German | #02 | 0.17 |
| 5 mm diameter | German | #02 | 0.104 |
| 5 mm diameter | German | #02 | 0.533 |
| 5 mm diameter | German | #02 | 0.267 |
| 5 mm diameter | German | #02 | 0.582 |
| 5 mm diameter | German | #02 | 2.693 |
| 5 mm diameter | German | #02 | 1.45 |
| 5 mm diameter | German | #02 | 1.59 |
| 5 mm diameter | German | #02 | 0.544 |
| 5 mm diameter | German | #02 | 4.84 |
| 5 mm diameter | German | #02 | 1.29 |
| 5 mm diameter | German | #02 | 0.503 |
| 5 mm diameter | German | #02 | 0.191 |
| 5 mm diameter | German | #02 | 0.057 |
| 5 mm diameter | German | #02 | 0.096 |
| 5 mm diameter | German | #02 | 1.89 |
| 5 mm diameter | German | #02 | 0.411 |
| 5 mm diameter | German | #02 | 3.169 |
| 2 mm diameter | German | #02 | 0.276 |
| 2 mm diameter | German | #02 | 0.217 |
| 2 mm diameter | German | #02 | 0.501 |
| 2 mm diameter | German | #02 | 0.621 |
| 2 mm diameter | German | #02 | 0.584 |
| 2 mm diameter | German | #02 | 2.166 |
| 2 mm diameter | German | #02 | 0.427 |
| 2 mm diameter | German | #02 | 0.347 |
| 5 mm diameter | German | #02 | 3.367 |
| 5 mm diameter | German | #02 | 3.01 |
| 5 mm diameter | German | #02 | 2.23 |

Biofilm: protein

| | | | Results Protein |
|---|---|---|---|
| PTFE channel | Soil | Formula | μg/cm² |
| 5 mm diameter | Biofilm | #02 | 0.63 |
| 5 mm diameter | Biofilm | #02 | 1.01 |
| 2 mm diameter | Biofilm | #02 | 1.79 |
| 2 mm diameter | Biofilm | #02 | 3.21 |

4^{th} Set of Experiments: Treatment with Chemistry (Formula #02) in Fuji G5/8 Surrogate

The surrogate comprises the following channels:
1 3 meters channel 2 mm diameter marked in green,
1 3 meters channel 2 mm diameter marked in blue,
1 3 meters channel 4.8 mm diameter marked in yellow.

The treatment comprises:
Flushing each channel with tap water at 1000 mPa for 15 seconds,
Filling each channel with chemistry (Formula #02),
Contact time: 2 minutes,
Flushing each channel with tap water at 1000 mPa for 30 seconds.

The results obtained are as detailed in the Table below:

| | | | | |
|---|---|---|---|---|
| German | TOC | green 3 | blue 3 | yellow 3 |
| soil | average | meters channel | meters channel | meters channel |
| | | 2 mm Ø | 2 mm Ø | 4.8 mm Ø |
| | Residual | 0.95 μg/cm² | 2.49 μg/cm² | 2.32 μg/cm² |
| | protein | | | |
| | average | | | |
| Austrian | TOC | 4.87 μg/cm² | 5.94 μg/cm² | 2.54 μg/cm² |
| soil | average | | | |
| | Residual | 0.35 μg/cm² | 1.66 μg/cm² | 0.44 μg/cm² |
| | protein | | | |
| | average | | | |

5^{th} Set of Experiments: Treatment with Chemistry (Formula #01 to #11) in PTFE Channels

In this set of experiments, different compositions are compared on German soil (worst case) in PTFE tubes (5 mm diameter).

The results obtained with formula #05 to formula #08 are presented below:

| | Formula #05 | Formula #06 | Formula #07 | Formula #08 |
|---|---|---|---|---|
| % by weight of the | | | | |
| composition | | | | |
| hydrogen peroxide | 7 | 7 | 7 | 14 |
| C12-14 alkyl | 12 | 12 | 12 | 12 |
| dimethyl amine | | | | |
| oxide | | | | |
| Sequestrant, MGDA | — | 8 | 4 | 4 |
| Phosphonate, HEDP | 1.02 | 4.86 | — | 3.09 |
| Phosphoric acid | — | — | 1.5 | — |
| fluidifying agent | 10 | 10 | 10 | 10 |
| RESULTS: | | | | |
| results after | a lot of blood | traces of blood | traces of blood | Visually clean, |
| treatment according | left in the | in the channel | in the channel | no residue |
| to the process | channel | | | |
| On 2 meters and | | | | |
| 5 mm diameter PTFE | | | | |
| Channel | | | | |

The tests above show:
the influence of the quantity of hydrogen peroxide on the quality of the final result. At concentrations of 7% of hydrogen peroxide, the soiled tubes are visually not clean after treatment;
that at an equivalent concentration of hydrogen peroxide, it is shown that the addition of sequestrant positively influences the final result in terms of cleaning.

The results obtained with formula #01 to formula #04 are presented in the Table below:

| % by weight of the | | | | |
|---|---|---|---|---|
| composition | Formula #01 | Formula #02 | Formula #03 | Formula #04 |
| hydrogen peroxide | 10.5 | 10.5 | 8.4 | 5.25 |
| alkyl dimethyl amine | 9.6 | 6 | 12 | 12 |
| oxide | | | | |
| Sequestrant, MGDA | 4 | 4 | 4 | 4 |
| Phosphonate, HEDP | 2.4 | 2.4 | 2.4 | 2.4 |
| fluidifying agent | 10 | 10 | 10 | 10 |
| results after | Visually clean, | Visually clean, | traces of blood | a lot of blood |
| treatment | no residue | no residue | in the channel | left |
| On 2 meters and | | | | |
| 5 mm diameter PTFE | | | | |
| Channel | | | | |

The tests above show the importance of having a minimal quantity of hydrogen peroxide on the final result (detergency with respect to German soiling).

With the formulas tested, at a concentration less than or equal to 8.4% hydrogen peroxide, the tubes tested were not visually clean at the end of the treatment.

### 6^{th} Set of Experiments: Stability of Hydrogen Peroxide in Different Compositions (Formula #09 to #12) Over Time

Different compositions that been tested regarding hydrogen peroxide stability over time.

The results are presented in the Table below:

| | #09 | #10 | #11 | #12 |
|---|---|---|---|---|
| hydrogen peroxide | 7 | 7 | 7 | 7 |
| alkyl dimethyl amine | 12 | 12 | 12 | 12 |
| oxide | | | | |
| Sequestrant, MGDA | 4 | 4 | 4 | 4 |
| Phosphonate, HEDP | 0.9 | 1.2 | 1.8 | 2.4 |
| fluidifying agent | 10 | 10 | 10 | 10 |
| RESULTS: | | | | |
| % of H₂O₂ loss after | 26% | 16% | 5.2% | 1.2% |
| 1 month at 40° c. | | | | |

The results show that the presence of a phosphonate (HEDP) influences the stability of the composition in terms of H₂O₂ loss. A loss of H₂O₂ after 1 month at 40° C. of less than 10% may be desired. For the composition tested, when the content in HEDP is less than 1.8%, the H₂O₂ loss is superior to 10%.

### 7^{th} Set of Experiments: Study of Viscosity of Formula #02

A composition of Formula #02 is prepared and is viscosity tested.

The viscosity is measured at 20° C. using a Brookfield DVIII Ultra rheometer (Sp61 module) between 75 and 275 rpm.

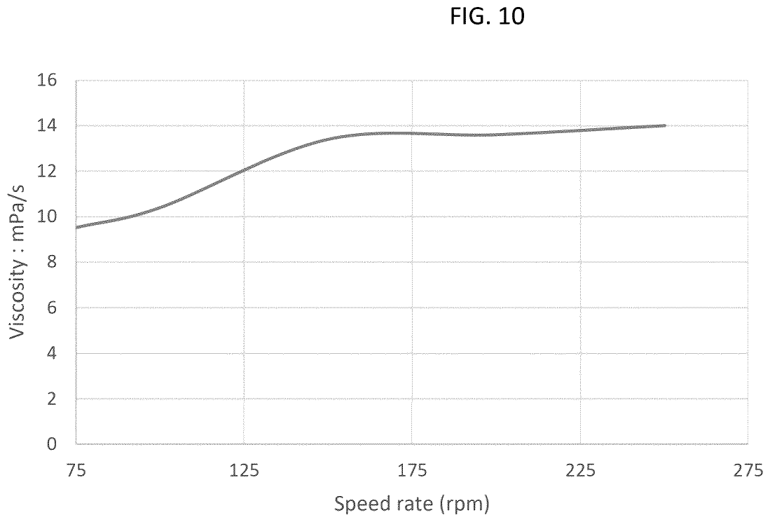

The results are shown in FIG. 10.

Example compositions of formulas #01 or #02 may for example be used as second fluid to operate block 1004 in any of example cleaning machines 4000, 5000, 6000, 7000, 8000 or 9000 and using a computer readable medium such as example storage 4072.

1. A method for cleaning a medical device, the method comprising:
coupling a lumen of the medical device to a cleaning machine, thereby providing a coupled lumen;
placing the medical device in a tank;
injecting a first volume of a first fluid from the cleaning machine, through the coupled lumen and into the tank for preliminary flushing of the lumen;
following the injection of the first volume of the first fluid, injecting a second fluid from the cleaning machine, through the coupled lumen and into the tank, wherein the second fluid differs from the first fluid;
following the injection of the second fluid, letting the second fluid sit in the lumen during a contact time period;
following the contact time period in which the second fluid sits in the lumen, injecting a second volume of the first fluid from the cleaning machine, through the coupled lumen and into the tank for rinsing the second fluid from the lumen;
following the contact time period in which the second fluid sits in the lumen, injecting a gas through the lumen; and
following the rinsing, maintaining the medical device in the tank during a soaking time, the tank containing a mixture of the first fluid and the second fluid.

2. The method according to claim 1, wherein the medical device is an endoscope.

3. The method according to claim 1, wherein the gas injection is following the rinsing and evacuates the first fluid from the lumen.

4. The method according to claim 1, wherein:
injecting of the first fluid for preliminary flushing of the lumens takes place at a pressure of more than 50 kPa and of less than 200 kPa; and
injecting of the first fluid for rinsing takes place at a pressure of more than 50 kPa and of less than 200 kPa.

5. The method according to claim 1, wherein the first fluid and the second fluid are at a temperature comprised between 5 degrees Celsius and 30 degrees Celsius.

6. The method according to claim 1, wherein:
the soaking time is of more than 30 s and of less than 600 s; and
the contact time is of more than 10 s and of less than 300 s.

7. The method according to claim 1, wherein the first fluid comprises more than 95% of water by weight.

8. The method according to claim 1, wherein the second fluid comprises an aqueous solution comprising hydrogen peroxide and one or more of a surfactant, a sequestrant, and an adjusting pH agent, and the second fluid has a pH of less than 8.

9. The method according to claim 1, wherein a ratio of a volume of the second fluid comprised in the tank during soaking to a total volume of the first fluid comprised in the tank during soaking is of less than 66%.

10. The method according to claim 1, further comprising brushing an external surface of the medical device during at least part of the soaking time.

11. The method according to claim 1, wherein the lumen has a diameter of more than 0.5 mm and of less than 6 mm.

12. The method according to claim 1, further comprising:
following rinsing of the lumen, placing the medical device in an automated endoscope reprocessor; and
performing an automated endoscope reprocessor cleaning on the medical device.

13. The method according to claim 1, wherein the medical device comprises a plurality of lumens, each lumen being submitted to the preliminary flushing, to the second fluid injection, to the contact time period, to the rinsing and to the gas injection.

14. A cleaning machine comprising:
a first fluidic connection to a supply of a first fluid;
a second fluidic connection to a supply of a second fluid different than the first fluid;
a gas connection to a supply of a gas;
a lumen connection;
a pump and a valve to selectively supply the first fluid, the second fluid, the gas, or a mixture of these, to the lumen connection; and
a control device including a processor, a storage and an instruction set configured to control the cleaning machine to:
inject a first volume of a first fluid through a lumen of a medical device coupled to the cleaning machine lumen connection and into the tank for preliminary flushing of the lumen;
following the injection of the first volume of the first fluid, inject a second fluid from the cleaning machine, through the lumen and into the tank;
following the injection of the second fluid, let the second fluid sit in the lumen during a contact time period;
following the contact time period in which the second fluid sits in the lumen, inject a second volume of the first fluid from the cleaning machine, through the coupled lumen and into the tank for rinsing the second fluid from the lumen;
following the contact time period in which the second fluid sits in the lumen, inject a gas through the lumen; and
following the rinsing, maintain the medical device in the tank during a soaking time, the tank containing a mixture of the first fluid and the second fluid.

15. The cleaning machine according to claim 14, wherein the lumen connection comprises a coupling block of the cleaning machine, and the coupling block comprising a plurality of lumen connections.

16. The cleaning machine according to claim 14, wherein the cleaning machine further comprises a container for the second fluid and a reserve of second fluid in the container, and wherein the second fluid comprises an aqueous solution comprising hydrogen peroxide and one or more of a surfactant, a sequestrant, and an adjusting pH agent, and the second fluid has a pH of less than 8.

17. A composition for cleaning of a medical device, wherein:
the composition is in the form an aqueous solution comprising hydrogen peroxide;
the composition has a pH of less than 8; and
the composition comprises one or more of a surfactant, a sequestrant, and an adjusting pH agent.

18. The composition according to claim 17, wherein the surfactant is selected from an alkyl dimethyl amine oxide, a sulfate, an ether sulfate, and a phosphate ester, and mixtures thereof.

19. The composition according to claim 17, wherein:
the sequestrant is selected from MGDA, IDS, GLDA, EDTA, NTA, and mixture thereof; and
the adjusting pH agent is selected from a phosphonate, an acid, and a mixture thereof.

20. The composition according to claim 17, comprising:
at least 5% of hydrogen peroxide;
between 1 and 10% of surfactant;
between 1 and 10% of sequestrant;
between 1 and 10% of adjusting pH agent; and
between 0 and 20% of fluidifying agent,
in each case where % is by weight of the composition.
